(19) United States
(12) Patent Application Publication
(10) Pub. No.: US 2018/0221570 A1
(43) Pub. Date: Aug. 9, 2018

(54) PUMPING APPARATUSES AND METHODS FOR FLUID INFUSION

(71) Applicant: VIDACARE LLC, Wayne, PA (US)

(72) Inventors: John F. MORGAN, III, San Antonio, TX (US); Robert W. TITKEMEYER, Shavano Park, TX (US)

(21) Appl. No.: 15/506,491

(22) PCT Filed: Aug. 25, 2015
(86) PCT No.: PCT/US2015/046655

§ 371 Date: Feb. 24, 2017

Related U.S. Application Data
(60) Provisional application No. 62/042,783, filed on Aug. 27, 2014.

(51) Int. Cl. A61M 5/142; F04B 43/12; A61M 5/168; A61M 5/158

(52) U.S. Cl. CPC A61M 5/14276; F04B 43/1253; A61M 5/16813; A61M 2205/0272; A61M 5/158; A61M 2210/02; A61M 2005/1585; A61M 5/14228

(57) ABSTRACT

This disclosure includes pumping apparatuses and methods of using the same. Some apparatuses include a pump housing having one or more walls extending from a proximal end to a distal end, a hub rotatably coupled to the proximal end, and a peristaltic pump coupled to the hub and configured to cause fluid flow through a flexible tube when the hub is rotated relative to the pump housing, where at least a portion of the pump is disposed within the pump housing. Some apparatuses include a rotor and an interior cam surface, where the rotor is configured to cause fluid flow through a flexible tube as the hub is rotated by compressing at least a portion of the flexible tube between the rotor and the interior cam surface. Some apparatuses are configured to be coupled in fixed relation to a housing of a driver.

### CROSS-REFERENCE TO RELATED APPLICATIONS

This application claims the benefit of priority of U.S. Provisional Patent Application No. 62/042,783, filed Aug. 27, 2014, which is hereby incorporated by reference in its entirety.

### BACKGROUND

### 1. Field of Invention

This disclosure relates generally to fluid infusion, such as into the intraosseous (IO) space, and more specifically, but not by way of limitation, to pumping apparatuses and methods for fluid infusion, such as fluid infusion into an IO space.

### 2. Description of Related Art

Vascular system access may be essential for treatment of many serious diseases, chronic conditions, and acute emergency situations. Yet, many patients experience extreme difficulty obtaining effective treatment because of an inability to obtain or maintain intravenous (IV) access. An IO space provides a direct conduit to a patent's vascular system and systemic circulation. Therefore, IO access is generally an effective route to administer a wide variety of drugs, other medications, IV fluids, and/or the like. Rapid IO access or emergency vascular access (EVA) offers great promise for almost any serious emergency that requires vascular access to administer life-saving drugs, other medications, fluids, and/or the like when traditional IV access is difficult or impossible.

Once IO access has been achieved, it may be desirable to infuse fluids (e.g., drugs, medications, IV fluids, and/or the like) into the IO space. Existing apparatuses for infusion, such as syringes, may not be capable of infusing a sufficient amount of fluid or performing the infusion within an effective amount of time.

### SUMMARY

Some embodiments of the present pumping apparatuses are configured, through a peristaltic pump configured to be coupled to a driver (such as a manually or electrically operated driver), to infuse fluids into, for example, an IO space. In some embodiments, the driver can also be used to insert an IO device into an IO space. Some embodiments are configured, through a fixable coupling between a pump housing and the driver, to achieve desirable functionality, such as, for example, single-handed pump operation.

Some embodiments of the present pumping apparatuses for fluid infusion comprise a pump housing having one or more walls extending from a proximal end to a distal end, a hub rotatably coupled to the proximal end of the pump housing, and a peristaltic pump coupled to the hub and configured to cause fluid flow through a flexible tube when the hub is rotated relative to the pump housing, at least a portion of the pump disposed within the pump housing. In some embodiments, the hub is configured to be removably coupled to a rotatable driveshaft of a driver. In some embodiments, the proximal end of the pump housing is configured to be coupled in fixed relation to a housing of a driver. In some embodiments, the pump housing has a transverse dimension that is between 5 to 10 times a transverse dimension of the rotatable driveshaft. In some embodiments, the driver is configured to insert an intraosseous device into bone or associated marrow.

In some embodiments, at least one of the one or more walls defines an interior cam surface and the peristaltic pump comprises a rotor configured to compress at least a portion of the flexible tube between the rotor and the interior cam surface. In some embodiments, the rotor is coupled to the hub and configured to cause fluid flow through the flexible tube as the hub is rotated. In some embodiments, the rotor comprises a non-circular cross-section. In some embodiments, the rotor comprises a rotor hub and a plurality of lobe members coupled to the rotor hub. In some embodiments, the plurality of lobe members is coupled in fixed relation to the rotor hub. In some embodiments, the plurality of lobe members is rotatable relative to the rotor hub. In some embodiments, the pump housing defines an internal recess adjacent the distal end of the pump housing, the recess configured to receive the plurality of lobe members.

Some embodiments comprise a sidewall having a barrel portion that extends longitudinally from the proximal end of the pump housing to define a longitudinal channel configured to receive at least a portion of the driver. In some embodiments, the sidewall comprises a trigger portion that extends at a non-parallel angle from the barrel portion, the trigger portion configured to receive a portion of a handle of the driver. In some embodiments, the sidewall defines an opening configured to allow access to a trigger of the driver when the apparatus is coupled to the driver.

In some embodiments, at least a portion of the pump housing is removable. In some embodiments, the distal end of the pump housing is removable form the proximal end of the pump housing.

In some embodiments, the pump housing defines an inlet and an outlet. In some embodiments, the inlet and outlet are defined on substantially a same side of the pump housing. In some embodiments, the inlet and the outlet are substantially co-planar. In some embodiments, at least a portion of the flexible tube extends through at least one of the inlet and the outlet of the pump housing.

Some embodiments comprise a releasable clamp configured to selectively block fluid communication through the flexible tube. Some embodiments comprise a flow regulator configured to be in fluid communication with the flexible tube. Some embodiments comprise a right angle valve configured to be in fluid communication with the flexible tube. Some embodiments comprise a needless valve configured to be in fluid communication with the flexible tube.

Some embodiments of the present methods for fluid infusion comprise coupling a rotatable driveshaft of a driver to a rotatable hub of a peristaltic pump, coupling a portion of the driver in fixed relation to a housing of the pump, and actuating the pump with the driver to cause fluid flow through a flexible tube in fluid communication with the pump. Some embodiments of the present methods for fluid infusion comprise coupling a rotatable driveshaft of a driver to a rotatable hub of a pump, the driver configured to insert an intraosseous device into bone or associated marrow, and actuating the pump to cause fluid flow through a flexible tube in fluid communication with the pump. Some embodiments comprise coupling a portion of the driver in fixed relation to a housing of the pump. Some embodiments comprise inserting an intraosseous device into bone or associated bone marrow with the driver and placing the intraosseous device into fluid communication with the pump.

The term “coupled” is defined as connected, although not necessarily directly, and not necessarily mechanically; two items that are “coupled” may be unitary with each other. The terms “a” and “an” are defined as one or more unless this disclosure explicitly requires otherwise. The term “substantially” is defined as largely but not necessarily wholly what is specified (and includes what is specified; e.g., substantially 90 degrees includes 90 degrees and substantially parallel includes parallel), as understood by a person of ordinary skill in the art. In any disclosed embodiment, the terms “substantially,” “approximately,” and “about” may be substituted with “within [a percentage] of” what is specified, where the percentage includes 0.1, 1, 5, and IO percent.

Further, a device or system that is configured in a certain way is configured in at least that way, but it can also be configured in other ways than those specifically described.

The terms “comprise” (and any form of comprise, such as “comprises” and “comprising”), “have” (and any form of have, such as “has” and “having”), “include” (and any form of include, such as “includes” and “including”), and “contain” (and any form of contain, such as “contains” and “containing”) are open-ended linking verbs. As a result, an apparatus that “comprises,” “has,” “includes,” or “contains” one or more elements possesses those one or more elements, but is not limited to possessing only those elements. Likewise, a method that “comprises,” “has,” “includes,” or “contains” one or more steps possesses those one or more steps, but is not limited to possessing only those one or more steps.

Any embodiment of any of the apparatuses, systems, and methods can consist of or consist essentially of—rather than comprise/include/contain/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

The feature or features of one embodiment may be applied to other embodiments, even though not described or illustrated, unless expressly prohibited by this disclosure or the nature of the embodiments.

Some details associated with the embodiments described above and others are described below.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers. The figures are drawn to scale (unless otherwise noted), meaning the sizes of the depicted elements are accurate relative to each other for at least the embodiment depicted in the figures.

FIG. 1A is a perspective view of one embodiment of the present pumping apparatuses.

FIGS. 1B-1E are bottom, top, back, and front views, respectively, of the apparatus of FIG. 1A.

FIG. 1F is a cross-sectional side view of the apparatus of FIG. 1A.

FIG. 1G is a cross-sectional and partially cutaway front view of the apparatus of FIG. 1A.

FIG. 1H is a perspective view of the apparatus of FIG. 1A, with a portion of the pump housing removed.

FIGS. 1I and 1J are side views depicting an example of attachment of the apparatus of FIG. 1A to a driver.

FIG. 2A is an exploded and partially cutaway side view of one example of an IO needle set or penetrator assembly.

FIG. 2B is a partial perspective view of a connector receptacle of the IO needle set of FIG. 2A.

FIGS. 3A and 3B are perspective and front views, respectively, of a rotor of the apparatus of FIG. 1A.

FIGS. 3C-3F are perspective, front, side, and back views, respectively, of a rotor hub of the rotor of FIG. 3A.

FIGS. 3G-3I are perspective, front, and side views, respectively, of a lobe member of the rotor of FIG. 2A.

FIG. 4A is a perspective view of a removable trigger guard for the apparatus of FIG. 1A.

FIG. 4B is a side view of the removable trigger guard of FIG. 4A installed on the apparatus of FIG. 1A.

FIG. 5 is a perspective view of the apparatus of FIG. 1A including related components.

### DETAILED DESCRIPTION OF ILLUSTRATIVE EMBODIMENTS

The present pumping apparatuses can be used for any type of infusion, such as, for example, IV infusion, IO infusion, infusion into a patient's stomach (e.g., enteral infusion) and/or any other internal organ, cavity, and/or the like. The present pumping apparatuses can be used to infuse fluids into and/or through any suitable location, space, and/or the like. Intraosseous infusion into an IO space is but one example of a suitable use for the present pumping apparatuses, is provided by way of illustration, and is in no way limiting.

Bone marrow typically includes blood, blood forming cells, and connective tissue disposed in an IO space or cavity surrounded by compact bone. Long bones such as the tibia typically have an elongated central cavity filled with yellow bone marrow and adipose or connective tissue. Such cavities may also be referred to as a “medullary cavity,” “bone marrow cavity,” and/or “intraosseous space.”

Compact bone disposed near an anterior or dorsal surface may be referred to as “anterior compact bone” or “anterior bone cortex.” Compact bone disposed farther from the dorsal or anterior surface may be referred to as “posterior compact bone” or “posterior bone cortex.”

Teachings of the present disclosure may be used to infuse fluids into patients, such as into an IO space of the patient, at a wide variety of insertion sites and target areas. Examples of insertion sites for an IO device to establish access with a patient's vascular system include the upper tibia proximate a patient's knee, the humeral head proximate a patient's shoulder, and the patient's sternum. Availability of multiple IO insertion sites and associated target areas in adjacent bone marrow have proven to be particularly important in applications such as emergency treatment of battlefield casualties or other mass casualty situations.

IO access may be used as a “bridge” for temporary fluid and/or drug therapy during emergency conditions until conventional IV sites can be found and used. Conventional IV sites often become available because fluids and/or medication provided via IO access may stabilize a patient and expand veins and other portions of a patient's vascular system. Pumping apparatuses and IO devices incorporating teachings of the present disclosure may become standard care for administering medications and fluids in situations when IV access is difficult or otherwise impossible.

IO access may be used as a “routine” procedure with chronic conditions, which substantially reduce or eliminate availability of conventional IV sites. Examples of such chronic conditions may include, but are not limited to, dialysis patients, patients in intensive care units, epilepsy patients, and/or the like. Pumping apparatuses and IO devices incorporating teachings of the present disclosure may be quickly and safely used to infuse fluids into an IO space, for example, in difficult cases, such as status epilepticus, to give medical personnel an opportunity to administer crucial medications, fluids, and/or the like.

Some apparatuses and methods incorporating teachings of the present disclosure may include using a first IO needle set having (e.g., a fifteen (15) gauge) cannula with a length of approximately fifteen (15) millimeters to establish vascular access for patients weighing between approximately three (3) kilograms and thirty nine (39) kilograms. A second IO needle set having a (e.g., a fifteen (15) gauge) cannula with an approximate length of twenty five (25) millimeters may be used to establish vascular access for patients weighing three (3) kilograms or greater. In other embodiments, a single size of IO needle set having a (e.g., a fifteen (15) gauge) cannula with an approximate length of twenty five (25) millimeters may be used to establish vascular access for patients weighing three (3) kilograms and greater.

The term “driver” may be used in this application to include any type of powered driver satisfactory for inserting an IO device such as a penetrator assembly, a catheter, an IO needle, an IO needle set, and/or the like into a selected portion of a patient's vascular system. Various techniques may be satisfactorily used to releasably engage or attach a pumping apparatus or IO device with a driver incorporating teachings of the present disclosure. For example, a wide variety of connectors and associated connector receptacles, fittings, and/or other types of connections with various dimensions and configurations may be satisfactorily used to releasably engage a pumping apparatus or IO device with a driver. A battery powered driver incorporating teachings of the present disclosure may be used to insert an IO device into a selected target area in ten (10) seconds or less, providing rapid IO access, which can then be used to infuse fluids into the IO space with a pumping apparatus. The reduced size and weight of drivers incorporating teachings of the present disclosure may accommodate use in emergency medical vehicles, in emergency crash carts at medical facilities, and/or in carrying in backpacks of military personnel deployed for extended periods of time in remote locations.

The term “fluid” may be used in this application to include liquids such as, but not limited to, blood, water, saline solutions, IV solutions, plasma, any mixture of liquids, particulate matter, dissolved medication, and/or drugs associated with biopsy and/or aspiration of bone marrow, and/or communication of fluids with bone marrow or other target sites. The term “fluid” may also be used in this patent application to include any body fluids and/or liquids containing particulate matter such as bone marrow and/or cells, which may be withdrawn from a target area.

The term “insertion site” may be used in this application to describe a location on a bone at which an IO device may be inserted or drilled into the bone and associated bone marrow. Insertion sites are generally covered by skin and soft tissue. The term “target area” refers to any location on or within biological material, such as the biological material of a human being.

The term “intraosseous device” or “IO device” may be used in this application to include, but is not limited to, any hollow needle, hollow drill bit, penetrator assembly, bone penetrator, catheter, cannula, trocar, stylet, inner penetrator, outer penetrator, IO needle, biopsy needle, aspiration needle, IO needle set, biopsy needle set, aspiration needle set, and/or the like, operable to access or provide access to an IO space or interior portions of a bone. Such IO devices may be formed, at least in part, from metal alloys such as 304 stainless steel and/or other biocompatible materials associated with needles and similar medical devices.

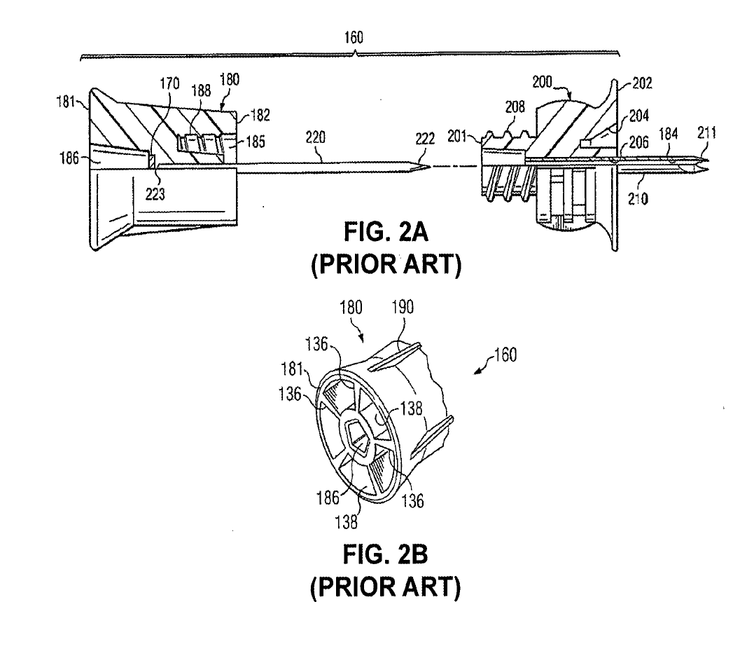

For some applications, an TO needle or TO needle set may include a connector with a trocar or stylet extending from a first end of the connector. A second end of the connector may be operable to be releasably engaged with a powered driver incorporating teachings of the present disclosure. An IO needle or IO needle set may also include a hub with a hollow cannula or catheter extending from a first end of the hub. A second end of the hub may include an opening sized to allow inserting the trocar through the opening and the attached hollow cannula. The second end of the hub may be operable to be releasably engaged with the first end of the connector. As previously noted, the second end of the connector may be releasably engaged with a powered driver. A wide variety of connectors and hubs may be used with an IO device incorporating teachings of the present disclosure. The present disclosure is not limited to connector 180 or hub 200 as shown in FIGS. 2A and 2B.

The IO device shown in FIGS. 2A and 2B is a prior art device, and the description of it is provided to give the reader context for the types of devices and components that can be used consistently with embodiments of the present pumping apparatuses, drivers, and kits, and/or the like.

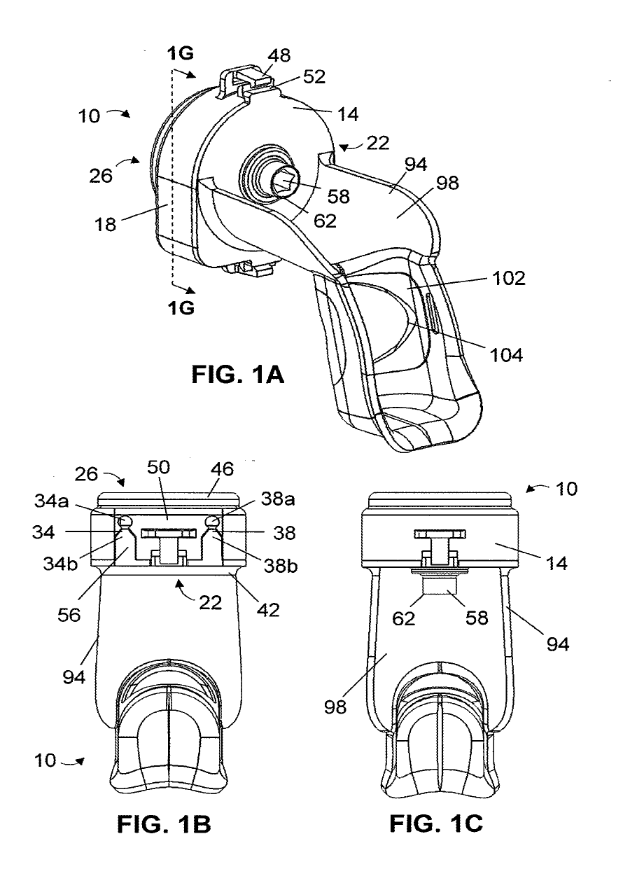

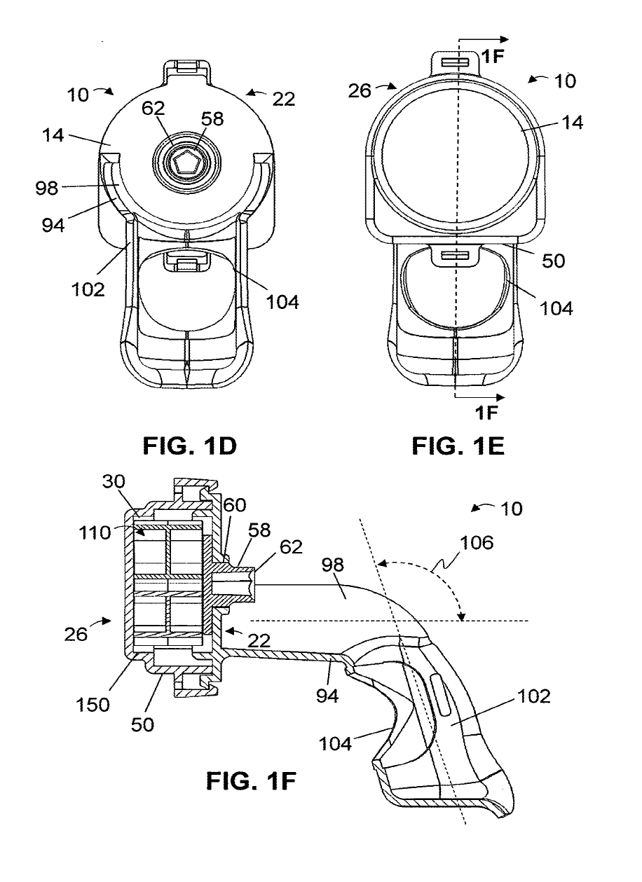

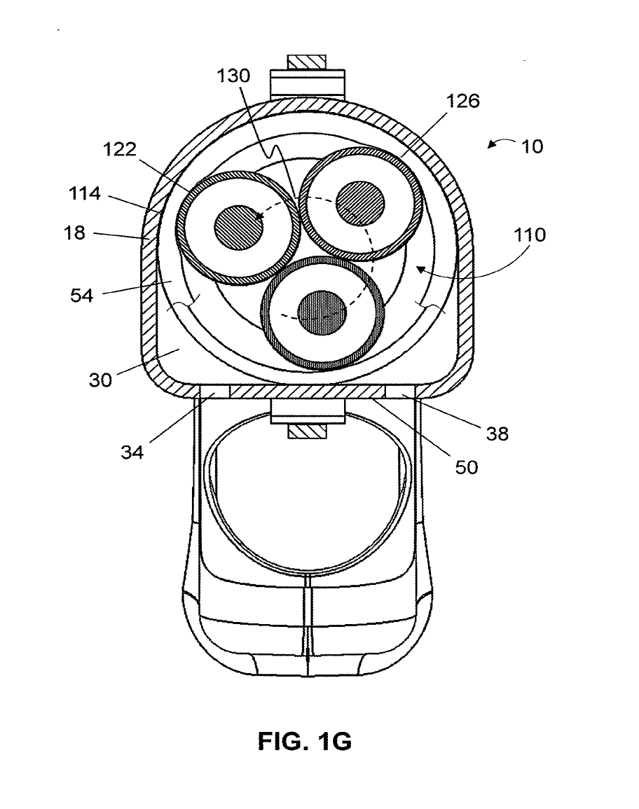

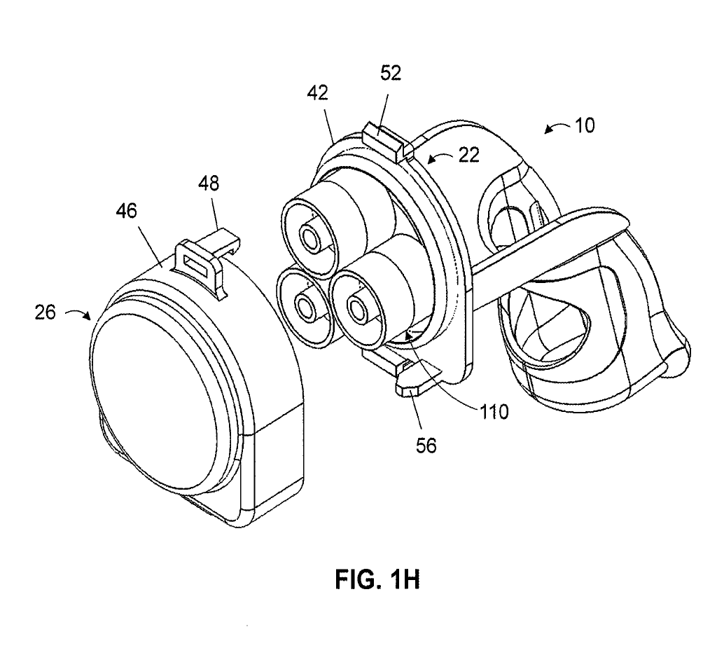

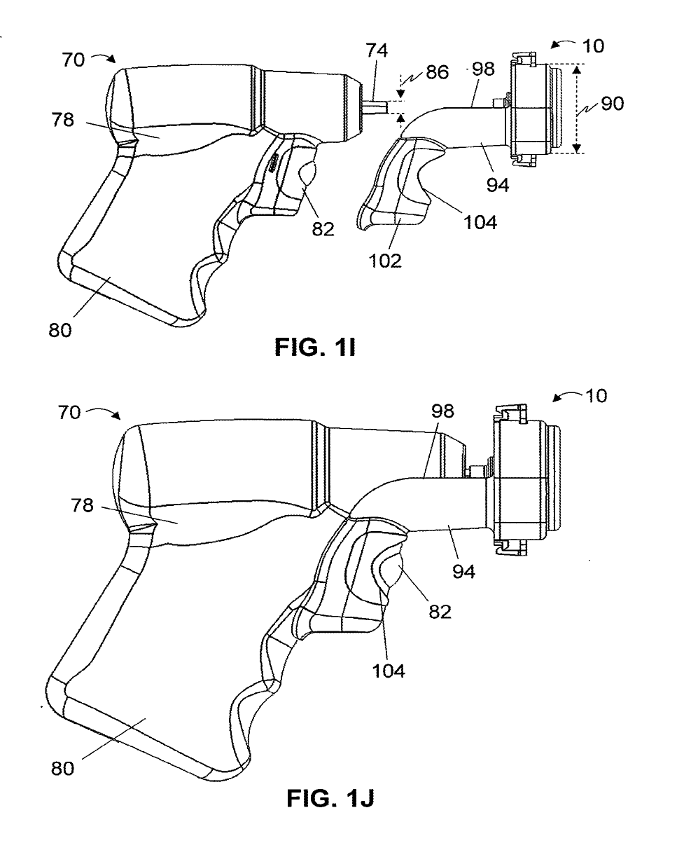

Referring now to the drawings, and more particularly to FIGS. 1A-1H, shown therein and designated by the reference numeral 10 is a first embodiment of the present pumping apparatuses. Apparatus 10 comprises a pump housing 14 having one or more walls 18 extending from a proximal end 22 to a distal end 26 (e.g., and defining an interior volume 30). In the depicted embodiment, proximal end 22 and distal end 26 are each substantially planar, the proximal end is substantially co-planar with the distal end, and the housing comprises a substantially round cross-section (e.g., as shown in FIG. 1G, with the exception of the portion of the housing that defines inlet 34 and outlet 38, described in more detail below). However, other embodiments can comprise any suitable shape, such as, for example, non-planar and/or non-coplanar proximal and distal ends, and/or generally triangular, square, rectangular, and/or otherwise polygonal cross-sections.

In the embodiment shown, at least a portion of pump housing 14 is removable (e.g., distal end 26 of pump housing 14 is removable from proximal end 22 of the pump housing, as shown in FIG. 1H). Particularly, in the depicted embodiment, housing 14 comprises a proximal portion 42 and a distal portion 46, which is removably coupled to the proximal portion (e.g., through snaps, fasteners, interlocking features, and/or the like). For example, in this embodiment, distal portion 46 comprises one or more latching members 48 (e.g., two latching members), which extend laterally from distal portion 46 in a proximal direction (e.g., away from distal end 26), and proximal portion 42 comprises one or more corresponding latching members 52, which are configured to receive and releasably couple to latching members 48 of distal portion 46 (e.g., and thus releasably couple proximal portion 42 to distal portion 46). In this way, pump housing 14 is configured to permit access to interior volume 30 (e.g., and associated pump components, described in more detail below), for example, to facilitate assembly, repair, cleaning, and/or the like of the present pumping apparatuses and related components. However, in some embodiments, the present pumping apparatuses can be configured such that proximal portion 42 is non-removably coupled to and/or is unitary with distal portion 46. For example, proximal portion 42 and distal portion 46 may be adhered (e.g., glued), welded (e.g., ultrasonically), attached through single-use attachment features (e.g., that are configured to break during detachment), and/or the like to one another. In this way, the present pumps can be configured to be single-use and/or non-serviceable (e.g., to promote sterility, prevent cross-contamination, and/or the like).

In the embodiment shown, pump housing 14 defines an inlet 34 and an outlet 38 (e.g., openings, each in direct communication with interior volume 30). In this embodiment, inlet 34 and outlet 38 are defined on substantially a same side of the housing (e.g., as shown). For example, in the depicted embodiment, inlet 34 and outlet 38 are each defined by a single planar wall 50 (e.g., inlet 34 and outlet 38 are substantially co-planar). In this way, a flexible tube 54 can be connected to and/or disposed through inlet 34 and/or outlet 38, while reducing the risk of the flexible tube becoming tangled, damaged, obtrusive to operation of the pumping apparatus, and/or inadvertently detached from the pump housing during operation. In some embodiments, at least a portion of flexible tube 54 extends from interior volume 30 and through at least one of inlet 34 and outlet 38. For example, the inlet and the outlet can each comprise a generally circular or rounded portion, 34a and 38a, respectively, which can be configured to receive flexible tube 54 (e.g., which may comprise a circular cross-section). In this embodiment, the inlet and the outlet each comprise a tapered portion, 34b and 38b, respectively, in communication with the respective circular or rounded portion and extending proximally through distal portion 46 of housing 14. In this way, flexible tube 54 can be installed and/or secured within housing 14, for example, by grasping detached distal portion 46, pressing the flexible tube laterally through tapered portion 34b and/or 38b, and into circular or rounded portion 34a and/or 38a, thus securing the flexible tube relative to the inlet and/or outlet. In the embodiment shown, proximal portion 42 comprises one or more protrusions 56, which can be received by and/or within distal portion 46 (e.g., by and/or within tapered portions 34b and/or 38b, as shown in FIG. 1B). In this way, one or more protrusions 56 can facilitate locating the distal portion relative to the proximal portion (e.g., during assembly) and/or securing flexible tube 54 relative to housing 14 (e.g., to prevent flexible tube 54 from moving relative to housing 14 during, for example, pump operation). In some embodiments, the inlet and/or the outlet can comprise a connector (e.g., a nipple), which extends into and/or away from interior volume 30, which can be suitable for attaching tubing.

In this embodiment, pumping apparatus 10 comprises a hub 58 rotatably coupled to proximal end 22 of pump housing 14 (e.g., and a portion of hub 58 can be extend through and/or into proximal end 22 and/or be disposed within interior volume 30, as shown). For example, in the depicted embodiment, hub 58 comprises a bearing surface 60, which is sized to rotatably rest within an opening defined by proximal end 22 (e.g., as shown). In this way, bearing surface 60 can support hub 58 as the hub rotates relative to housing 14. However, in other embodiments, hub 58 can be rotatably coupled and/or supported relative to pump housing 14 through any suitable structure, such as, for example, through bushings, bearings, other bearing surfaces, and/or the like. In the embodiment shown, hub 58 (e.g., and/or a proximal portion 62 thereof) is configured to be removably coupled to a rotatable driveshaft 74 of a driver 70 (e.g., such that driver 70 may rotate hub 58 and actuate a peristaltic pump coupled to hub 58, described in more detail below, to cause fluid flow through flexible tube 54).

Driver 70 is powered (e.g., electrically, by a battery), having a generally pistol-shaped housing 78, rotatable driveshaft 74, and a trigger 82 (e.g., to activate an electrical motor of driver 70 and rotate driveshaft 74). As shown, pump housing 14 can be configured (e.g., sized) to work effectively with driver 70. For example, in this embodiment, pump housing 14 has a transverse dimension 90 that is between 5 to 10 times a transverse dimension 86 of driveshaft 74. For a more detailed description of some suitable drivers for use with the present pumping apparatuses, see U.S. patent application Ser. No. 12/025,580, which is expressly incorporated by reference in its entirety, and more specifically ¶¶ 0046-0056 and FIGS. 1D-1E, 2A-2B, 3, and 5A-5D. Any driver described and/or referenced in this disclosure is provided only by way of example. In some embodiments, hub 58 may be configured to be driven manually, for example, by comprising and/or being configured to receive a handle, crank, lever, a driver comprising a hand crank configured to rotate a rotatable driveshaft, and/or the like.

Driver 70 may be configured to insert an IO device (e.g., IO needle set, penetrator assembly, or IO device 160) into bone or associated marrow (e.g., to provide IO access for infusion with pumping apparatus 10). For example, proximal portion 62 of hub 58 may be substantially similar to a connector (e.g., 180) and/or comprise a substantially similar coupling structure (e.g., first end 181 and/or opening 186 of connector 180) as a connector or hub of an IO device (e.g., 160). In this way, pumping apparatus 10 can be configured to infuse fluids into an IO space accessed by an IO device, without changing or substantially reconfiguring the driver (e.g., driveshaft 74 of driver 70 can be configured to be coupled to hub 58 as well as connector 180).

An example of a penetrator assembly 160 is shown in FIGS. 2A and 2B, and may include connector 180, associated hub 200, outer penetrator 210, and inner penetrator 220. Penetrator assembly 160 may include an outer penetrator such as a cannula, a hollow tube or hollow drill bit, and an inner penetrator such as a stylet or trocar. Various types of stylets and/or trocars may be disposed within an outer penetrator. For some applications, outer penetrator or cannula 210 may be described as a generally elongated tube sized to receive inner penetrator or stylet 220 therein. Portions of inner penetrator 220 may be disposed within longitudinal passageway 184 extending through outer penetrator 210. The outside diameter of inner penetrator 220 and the inside diameter of longitudinal passageway 184 may be selected such that inner penetrator 220 may be slidably disposed within outer penetrator 210.

Metallic disc 170 may be disposed within opening 186 for use in releasably attaching connector 180 with a magnet disposed on a distal end of driveshaft 74 (e.g., or an otherwise magnetic driveshaft 74). End 222 of inner penetrator 220 may be spaced from metallic disc 170 with insulating or electrically nonconductive material disposed therebetween. In some embodiments, metallic disk 170 may be magnetic, and driveshaft 74 may be metallic. Such magnetic coupling can provide audible and/or tactile feedback to a user when assembling the present components (e.g., IO devices, pumping apparatuses, drivers, and/or the like), mitigate the risk of inadvertent separation of the assembled components during use, and/or the like. As discussed above, hub 58 (e.g., proximal portion 62) may be substantially similar to a connector (e.g., 180) and/or comprise a substantially similar coupling structure (e.g., first end 181 and/or opening 186 of connector 180) as a connector or hub of an IO device (e.g., 160). For example, hub 58 (e.g., proximal portion 62) of pumping apparatus 10 may comprise a metallic and/or magnetic portion (e.g., a disk) configured to magnetically engage a metallic and/or magnetic portion of driveshaft 74.

Tip 211 of outer penetrator 210 and/or tip 222 of inner penetrator 220 may be operable to penetrate bone and associated bone marrow. The configuration of tips 211 and/or 222 may be selected to penetrate a bone or other body cavities with minimal trauma. First end or tip 222 of inner penetrator 220 may be trapezoid shaped and may include one or more cutting surfaces. In some embodiments, outer penetrator 210 and inner penetrator 220 may be ground together as one unit during an associated manufacturing process. Providing a matching fit allows respective tips 211 and 222 to act as a single drilling unit, which facilitates insertion and minimizes damage as portions of penetrator assembly 160 are inserted into a bone and associated bone marrow. Outer penetrator 210 and/or inner penetrator 220 may be formed from stainless steel, titanium, and/or other materials of suitable strength and durability to penetrate bone.

Hub 200 may be used to stabilize penetrator assembly 160 during insertion of an associated penetrator into a patient's skin, soft tissue, and adjacent bone at a selected insertion site. First end 201 of hub 200 may be operable for releasable engagement or attachment with associated connector 180. Second end 202 of hub 200 may have a size and configuration compatible with an associated insertion site for outer penetrator 210. The combination of hub 200 with outer penetrator 210 may sometimes be referred to as a “penetrator set” or “intraosseous needle.”

Connector 180 and attached inner penetrator 220 may be releasably engaged with hub 200 by Luer type fittings, threaded connections, and/or other suitable fittings formed on first end 201 of hub 200. Outer penetrator 210 extends from second end 202 of hub 200.

For some applications connector 180 may be described as a generally cylindrical tube defined in part by first end 181 and second end 182. The exterior of connector 180 may include an enlarged tapered portion adjacent to end 181. A plurality of longitudinal ridges 190 may be formed on the exterior of connector 180 to allow an operator to grasp associated penetrator assembly 160 during attachment with a driveshaft. Longitudinal ridges 190 also allow connector 180 to be grasped for disengagement from hub 200 when outer penetrator 210 has been inserted into a bone and associated bone marrow.

Second end 182 of connector 180 may include opening 185 sized to receive first end 201 of hub 200 therein. Threads 188 may be formed in opening 185 adjacent to second end 182 of connector 180. Threads 188 may be used in releasably attaching connector 180 with threaded fitting 208 adjacent to first end 201 of hub 200.

First end 201 of hub 200 may include a threaded connector 208 and/or other suitable fittings formed on the exterior thereof. First end 201 may have a generally cylindrical pin-type configuration compatible with releasably engaging second end or box end 182 of connector 180.

For some applications second end 202 of hub 200 may have the general configuration of a flange. Angular slot or groove 204, which can be formed in end 202, can be sized to receive one end of protective cover or needle cap.

For some applications a penetrator assembly may include only a single, hollow penetrator. For other applications a penetrator assembly may include an outer penetrator such as a cannula, a hollow needle, or a hollow drill bit, and an inner penetrator such as a stylet, trocar, or other removable device disposed within the outer penetrator. Penetrator 210 is one example of a single, hollow penetrator or cannula.

The size of a penetrator may vary depending upon the intended application for the associated penetrator assembly. Penetrators may be relatively small for pediatric patients, medium size for adults, and large for oversize adults. By way of example, a penetrator may have a length greater than any one of or between any two of: five (5) mm, forty five (45) mm, one hundred and fifty two (152) mm, or larger. The diameter of a penetrator may range from eighteen (18) gauge to ten (10) gauge. The length and diameter of the penetrator used in a particular application may depend on the size of a bone to which the apparatus may be applied. Penetrators may be provided in a wide variety of configurations depending upon intended clinical purposes for insertion of the associated penetrator. For example, there may be one configuration for administering drugs and/or fluids to a patient's bone marrow and an alternative configuration for sampling bone marrow and/or blood from a patient. Other configurations may be appropriate for bone and/or tissue biopsy.

First end 181 of connector of 180 may include opening 186 sized to receive portions driveshaft 74 therein. A plurality of webs 136 may extend radially outward from opening 186. Webs 136 may cooperate with each other to form a plurality of openings 138 adjacent to first end 181. Opening 186 and openings 138 may cooperate with each other to form portions of a connector receptacle operable to receive respective portions of a connector (not expressly shown) therein.

Referring back to FIGS. 1A-1J, pumping apparatus 10 can be configured to be coupled in fixed relation to housing 78 of driver 70 (e.g., when driveshaft 74 is coupled to hub 58). In this way, pumping apparatus 10 can be actuated by a driver while avoiding rotation of pump housing 14 relative to driver 70 (e.g., facilitating single-handed operation). For example, in the embodiment shown, pump housing 14 comprises a sidewall 94 having a barrel portion 98, which extends longitudinally from proximal end 22 of the pump housing to define a longitudinal channel configured to receive at least a portion of driver 70 (e.g., as shown in FIGS. 1I and 1J). Sidewall 94 can be configured to be secured to housing 78 through any suitable structure, such as, for example, snaps, fasteners, interlocking features disposed on pumping apparatus 10 and/or driver 70, and/or the like. In the embodiment shown, sidewall 94 comprises a trigger portion 102, which extends generally at a non-parallel angle 106 from barrel portion 98 and is configured to receive a portion of a handle 80 of the driver. Sidewall 94 can be configured to allow operation of driver 70 when pumping apparatus 10 is coupled to the driver, for example, in this embodiment, sidewall 94 (e.g., trigger portion 102) defines an opening 104 configured to allow access to a trigger 82 of the driver when pumping apparatus 10 is coupled to the driver.

As mentioned above, in the embodiment shown, pumping apparatus 10 comprises a peristaltic pump 110 coupled to hub 58 (e.g., and at least partially disposed within interior volume 30). Peristaltic pump 110 comprises an interior cam surface 114 defined on an interior portion of at least one of the one or more walls 18 (e.g., a surface of a wall 18 facing interior volume 30). As shown, in this embodiment, interior cam surface 114 is substantially smooth and comprises a substantially cylindrical shape. However, in other embodiments, interior cam surface 114 can comprise ridges, bumps, protrusions, and/or the like (e.g., which may enhance peristalsis, described below). In this embodiment, peristaltic pump 110 comprises a rotor 118 configured to compress at least a portion of flexible tube 54 between the rotor and interior cam surface 114 (e.g., as shown in FIG. 1G) (e.g., at the depicted orientation of rotor 118 relative to interior cam surface 114, at locations 122 and 126, for example, by way of rotor 118 comprising a non-circular cross-section). In this way, as rotor 118 rotates relative to housing 14 (e.g., along a direction indicated by arrow 130), flexible tube 54 is compressed and released at varying points along its length, resulting in peristalsis in and fluid flow through flexible tube 54 (e.g., from inlet 34 to outlet 38).

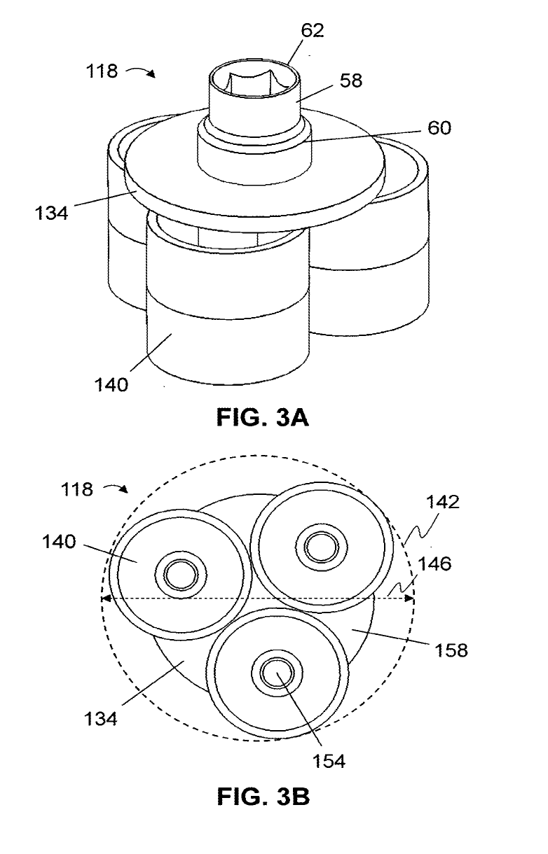

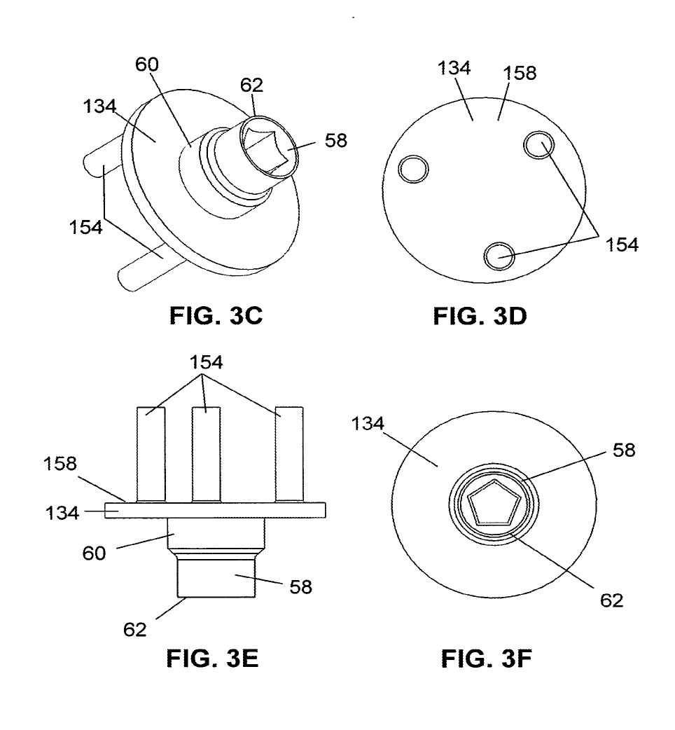

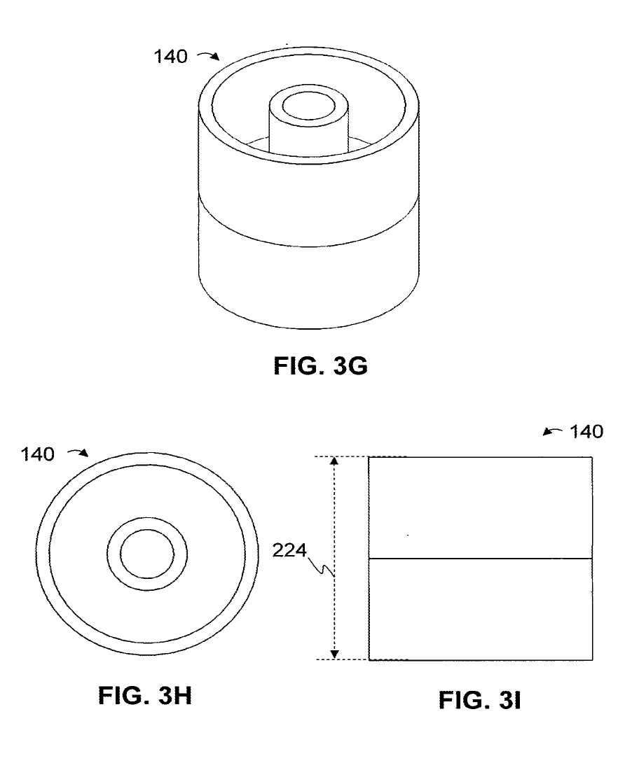

FIGS. 3A-3I depict various views of a rotor 118 (and associated components) of pumping apparatus 10. In the embodiment shown, rotor 118 comprises a rotor hub 134, which can be coupled to (e.g., in a rotatably fixed relationship) and/or unitary with hub 58. In the embodiment shown, rotor 118 comprises a plurality of lobe members 140, described in more detail below, coupled to rotor hub 134. In this embodiment, rotor 118 comprises three (3) lobe members 140; however, in other embodiments, the rotor can comprise any suitable number of lobe members, such as, for example, 1, 2, 3, 4, 5, 6, 7, 8, 9, 10, or more lobe members. In some embodiments, rotor 118 and/or rotor hub 134 can be sized to perform the function of lobe members 140 (e.g., by comprising protrusions that extend laterally from a rotational axis of the rotor and can function similarly to the lobe members) and the lobe members may be omitted. In the embodiment shown, during rotation, rotor 118 circumscribes a circle 142 having a transverse dimension (e.g., diameter) 146. In some embodiments (e.g., 10) pump housing 14 defines an internal recess 150 (e.g., forming part of interior volume 30) adjacent distal end 26 and configured to receive rotor 118 and/or lobe members 140 (e.g., by having a transverse dimension corresponding to transverse dimension 146). In this way, pump housing 14 can be configured to control lateral deflection of rotor 118 (and associated components) and/or hub 58 as peristaltic pump 110 is actuated.

In the embodiment shown, lobe members 140 of rotor 118 are coupled to rotor hub 134 such that the lobe members are each permitted to rotate relative to the rotor hub. For example, lobe members 140 can each be rotatably disposed on a different one of a plurality a protrusions 154 (e.g., axles), which each extend longitudinally from a second end 158 of rotor hub 134. In this way, as rotor 118 rotates relative to pump housing 14 and compresses flexible tube 54 (e.g., through contact with lobe members 140), the lobe members can rotate relative to rotor hub 134 and mitigate shear stresses between the lobe members and the flexible tube (e.g., extending the life of the flexible tube and/or permitting smooth operation). However, in other embodiments, lobe members 140 can be fixed relative to or unitary with rotor hub 134.

In the embodiment shown, lobe members 140 are each substantially cylindrical (e.g., having a substantially circular cross-section as shown in FIG. 3H) and are each substantially similar to one another. However, lobe members of other embodiments may not be uniform, and/or can comprise any suitable shape, such as, for example, square, rectangular, triangular, otherwise polygonal, circular, elliptical, otherwise rounded, and/or the like. As shown, each lobe member has a height 224 such that the lobe member spans a majority of interior volume 30 in a direction from proximal end 22 and through distal end 26 when rotor 118 is disposed within pump housing 14 (e.g., as shown in FIG. 1F). In this way, sufficient compression of flexible tube 54 between interior cam surface 114 and rotor 118 is facilitated, notwithstanding any lateral displacements of flexible tube 54 relative to interior cam surface 114.

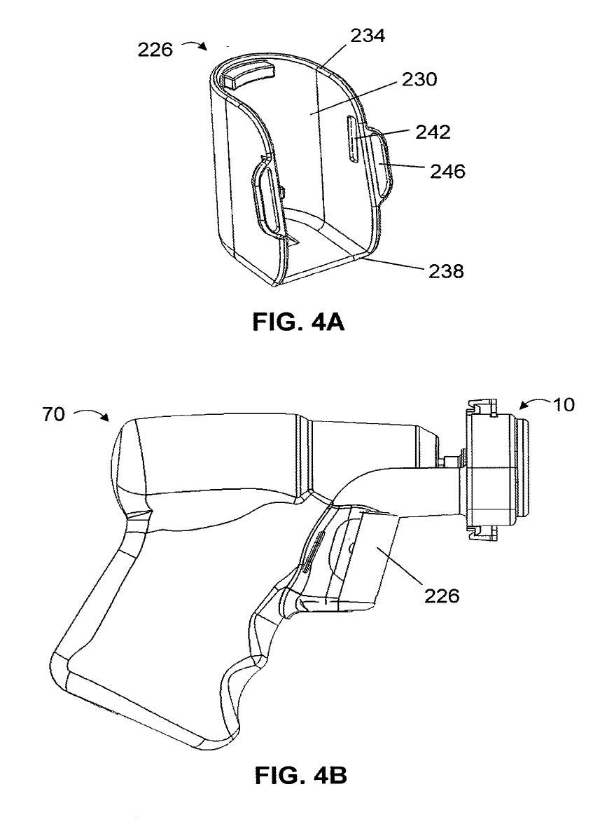

As shown in FIGS. 4A and 4B, some embodiments of the present pumping apparatuses comprise a removable trigger guard 226 (e.g., to prevent inadvertent operation of driver 70 and/or apparatus 10). In this embodiment, removable trigger guard 226 comprises a generally u-shaped channel 230, which extends from a first end 234 to a second end 238 (e.g., which may be closed, as shown, depending on the configuration of the driver and/or pumping apparatus). Removable trigger guard 226 can be configured to be coupled to a pumping apparatus (e.g., 10, at trigger portion 102 of sidewall 94 to cover opening 104) and/or a driver (e.g., 70), for example, through openings or recesses 242 defined within channel 230 and/or through trigger guard 226 and configured to receive corresponding protrusions of the pumping apparatus and/or driver. In this embodiment, removable trigger guard 226 comprises two wings 246, which can be configured to facilitate removal of trigger guard 226, for example, to access trigger 82 of driver 70 when pumping apparatus 10 is attached.

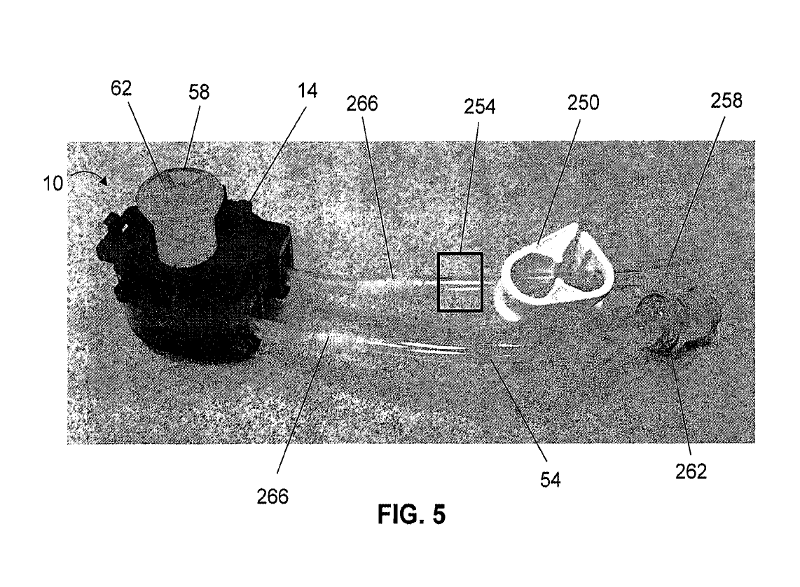

FIG. 5 is a perspective view of the pumping apparatus of FIG. 1A (without sidewall 94), with related components. Pumping apparatuses of the present disclosure are suitable for use with any number of connectors, valves, clamps, regulators, other components, and/or the like. The following components are provided only by way of example. In the embodiment shown, a releasable clamp 250 is coupled to and configured to selectively block fluid communication through flexible tube 54. In this embodiment, a flow regulator 254 can be configured to be in fluid communication with the flexible tube (e.g., which can be configured to even out pressure fluctuations that may occur in fluid flow during operation of peristaltic pump 110). As discussed above, pumping apparatus 10 can be configured to infuse fluids into an IO space (e.g., through an IO device in communication with the IO space). For example, in this embodiment, a valve (e.g., right angle valve 258 and/or a needleless valve 262) is configured to allow fluid communication to and/or from pumping apparatus 10 to and/or from an IO device (e.g., 160, for example, through connection to threaded fitting 208).

As shown, in this embodiment, one or more tube couplers 266 (e.g., barbed tube couplers) can be coupled to and/or between portions of flexible tube 54. In this way, the diameter (e.g., inner diameter) of the flexible tube can be varied throughout the system (e.g., between pump 10, flow regulator 254, right angle valve 258, needleless valve 262, portions of flexible tube 54, other components, and/or the like). For example, tube couplers 266 can be placed at any suitable location within the system and can allow the connection of flexible tube(s) having various inner diameter(s). In particular, in this embodiment, portions of flexible tube 54 between pump 10 and right angle valve 258 and between pump 10 and needleless valve 262 have a larger inner diameter than portions of flexible tube 54 within and/or proximate to pump 10. In this way, fluid friction within the system can be controlled, adjusted, and/or reduced (e.g., which can allow a relatively longer portion of flexible tube 54 to be disposed within pump 10, without substantially adversely affecting fluid friction through the system).

Some of the present methods for fluid infusion comprise coupling a rotatable driveshaft (e.g., 74) of a driver (e.g., 70) to a rotatable hub (e.g., 58) of a peristaltic pump (e.g., 110), coupling a portion of the driver (e.g., housing 78, handle 80, and/or the like) in fixed relation to a housing (e.g., 14) of the pump, and actuating the pump with the driver to cause fluid flow through a flexible tube (e.g., 54) in fluid communication with the pump. In some methods the driver is configured to insert an IO device (e.g., 160) into bone or associated marrow. Some methods comprise inserting an TO device into bone or associated marrow with the driver and placing the IO device into fluid communication with the pump.

Pumping apparatuses (e.g., 10) of the present disclosure can be included as part of a kit. For example, kits of the present disclosure can include pumping apparatus(es) (e.g., 10), IO needle set(s) (e.g., 160), flexible tubing (e.g., 54), driver(s) (e.g., 70), and/or the like. Some kits may be sterile.

The above specification and examples provide a complete description of the structure and use of illustrative embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the methods and systems are not intended to be limited to the particular forms disclosed. Rather, they include all modifications and alternatives falling within the scope of the claims, and embodiments other than the one shown may include some or all of the features of the depicted embodiment. For example, elements may be omitted or combined as a unitary structure, and/or connections may be substituted. Further, where appropriate, aspects of any of the examples described above may be combined with aspects of any of the other examples described to form further examples having comparable or different properties and/or functions, and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

1-30. (canceled)

31. A pumping apparatus for fluid infusion, the apparatus comprising:
a pump housing having one or more walls extending from a proximal end to a distal end;
a hub rotatably coupled to the proximal end of the pump housing; and
a peristaltic pump coupled to the hub and configured to cause fluid flow through a flexible tube when the hub is rotated relative to the pump housing, at least a portion of the pump disposed within the pump housing;
where the proximal end of the pump housing is configured to be coupled in fixed relation to a housing of a driver.

32. The apparatus of claim 31, where the hub is configured to be removably coupled to a rotatable driveshaft of the driver.

33. The apparatus of claim 32, where the pump housing has a transverse dimension that is between 5 to 10 times a transverse dimension of the rotatable driveshaft.

34. The apparatus of claim 31, where the driver is configured to insert an intraosseous device into bone or associated bone marrow.

35. The apparatus of claim 31, where at least one of the one or more walls defines an interior cam surface and the peristaltic pump comprises a rotor configured to compress at least a portion of the flexible tube between the rotor and the interior cam surface.

36. The apparatus of claim 35, where the rotor comprises a non-circular cross-section.

37. The apparatus of any of claim 35, where the rotor comprises a rotor hub and a plurality of lobe members coupled to the rotor hub.

38. The apparatus of claim 37, where the pump housing defines an internal recess adjacent the distal end of the pump housing, the recess configured to receive the plurality of lobe members.

39. The apparatus of claim 31, further comprising a sidewall having a barrel portion that extends longitudinally from the proximal end of the pump housing to define a longitudinal channel configured to receive at least a portion of the driver.

40. The apparatus of claim 39, where the sidewall comprises a trigger portion that extends at a non-parallel angle from the barrel portion, the trigger portion configured to receive a portion of a handle of the driver.

41. The apparatus of claim 31, where at least a portion of the pump housing is removable.

42. The apparatus of claim 41, where the distal end of the pump housing is removable from the proximal end of the pump housing.

43. The apparatus of claim 31, where the pump housing defines an inlet and an outlet.

44. The apparatus of claim 43, where the inlet and outlet are defined on substantially a same side of the pump housing.

45. The apparatus of claim 43, where the inlet and the outlet are substantially co-planar.

46. The apparatus of claim 43, where at least a portion of the flexible tube extends through at least one of the inlet and the outlet of the pump housing.

47. The apparatus of claim 31, comprising a releasable clamp configured to selectively block fluid communication through the flexible tube.

48. The apparatus of claim 31, comprising a flow regulator configured to be in fluid communication with the flexible tube.

49. A method for fluid infusion, the method comprising:
coupling a rotatable driveshaft of a driver to a rotatable hub of a pump, the driver configured to insert an intraosseous device into bone or associated marrow; and
actuating the pump to cause fluid flow through a flexible tube in fluid communication with the pump.

50. The method of claim 49, comprising:
inserting an intraosseous device into bone or associated bone marrow with the driver; and
placing the intraosseous device into fluid communication with the pump.
